(19) United States
(12) Patent Application Publication
(10) Pub. No.: US 2022/0051809 A1
(43) Pub. Date: Feb. 17, 2022

(54) SYSTEM OF PREDICTING RISKS WITH BIOMEDICAL DATA, METHOD OF PREDICTING RISKS WITH BIOMEDICAL DATA, AND NON-TRANSIENT STATE COMPUTER-READABLE STORAGE MEDIUM

(71) Applicant: NATIONAL CHUNG SHAN INSTITUTE OF SCIENCE AND TECHNOLOGY, Taoyuan City (TW)

(72) Inventors: HO-HUI HSIEH, Taoyuan City (TW); HSING-RONG CHAO, Taoyuan City (TW); SHYH-JIAN TANG, Taoyuan City (TW)

(21) Appl. No.: 16/992,139

(22) Filed: Aug. 13, 2020

(51) Int. Cl. G16H 70/60; G16H 10/60

(52) U.S. Cl. CPC G16H 70/60; G16H 10/60

(57) ABSTRACT

A system of predicting risks with biomedical data includes a data collecting unit for receiving a plurality of data before arranging and integrating each of the plurality of data to create a plurality of medical data; a data processing unit for receiving the plurality of medical data, performing a data processing process on each of the plurality of medical data, and arranging and integrating each of the processed data to create a plurality of risk determination information; and a judgment unit comprising a storage unit and a prediction unit, wherein the plurality of risk determination information is stored in the storage unit, and the prediction unit performs estimation according to the plurality of risk determination information to generate a risk evaluation information.

### BACKGROUND OF THE INVENTION

### 1. Field of the Invention

The present disclosure relates to a system and method of predicting risks with biomedical data and a non-transient state computer-readable storage medium, and in particular to a system and method of predicting health risks with biomedical data and a non-transient state computer-readable storage medium.

### 2. Description of the Related Art

Chronic diseases, such as cardiovascular diseases, can be diagnosed with medical examinations and thus treated earlier. For instance, cardiovascular diseases are diagnosed with medical examinations like history taking, chest x-ray, blood test, electrocardiogram, cardiac-CT, myocardial perfusion scan, and angiography. Advanced examinations will not be recommended until symptoms are discovered with the aforesaid basic examinations.

Health examinations, which are intended to achieve “earlier diagnosis, earlier treatment” and emphasize the concept “prevention is better than cure,” are currently regarded as a scientific solution to life extension. However, conventional health checkups include basic examinations but not advanced examinations; as a result, physicians are required to give diagnoses according to the results of the basic examinations. However, owing to heavy traffic of the health checkups, the physicians compiling health checkup reports have too limited access to related information (which is seldom sufficiently disclosed) to identify high-risk patients and treat these patients earlier. In view of this, a computer-aided diagnosis system is an important tool that assists physicians in making decisions.

Conventional computer-aided diagnosis systems mostly use statistical models to estimate trends, and their software development is based on long-term follow-up data of plenty patients to estimate disease risks in accordance with data statistics. For example, Cardiovascular Disease Risk Evaluation Rules, posted on the official Website of the National Heart Lung and Blood Institute, the National Institutes of Health (NIH), the United States, based on the Framingham heart study (since 1948), and highly regarded by medical professionals, apply to cardiovascular disease risks like atherosclerosis, coronary syndrome, heart failure, myocardial infarction, hypertension.

Another type of computer-aided diagnosis systems are expert systems, whose software computes causal relationships according to expert-defined program rules. If the program rules are defined in great detail and in great amount, the software performance is likely to approximate to the human experts' in terms of diagnoses. For example, a diagnostic decision support system (Iliad), which took the University of Utah eight years to develop, contains information about 2,200 diseases and over 10,000 signs and symptoms.

The latest computer-aided diagnosis systems operate by artificial intelligence (AI), including machine learning and deep learning. The systems rely on abundant data in training AI models with a view to allowing the AI models to spontaneously attain diagnosis performance that approximates to the human experts'. Furthermore, the systems dispense with human-defined program rules and are exemplified by the chronic coronary syndromes (CCS) evaluation software approved by the FDA in 2018.

Machine learning decision support systems require data with stable quality to perform computation. However, the quality of the data can alter for extrinsic reasons, such as equipment ageing, equipment changes, personnel changes, operation mistakes, and data loss, leading to model prediction errors. In this regard, hospital data quality is usually governed by internal quality assurance rules. However, not only do quality assurance rules vary from medical institution to medical institution, but related rules may also evolve with time; thus, collection of data with equal quality is difficult. Therefore, to equalize data quality and eliminate negative effects of bad data on prediction models, the machine learning decision support systems come with a data quality pre-processing function.

### BRIEF SUMMARY OF THE INVENTION

An objective of the present disclosure is to provide a system of predicting risks with biomedical data, which corrects data and enters a missing value with an algorithm to render the data consistent and enhance prediction accuracy, so as to eliminate the effect of bad data on prediction accuracy of artificial intelligence models and thereby enhance the accuracy in predicting disease risks from personal health records.

To achieve at least the above objective, the present disclosure provides a system of predicting risks with biomedical data, comprising: a data collecting unit for receiving a plurality of data and then arranging and integrating each of the plurality of data to create a plurality of medical data; a data processing unit for receiving the plurality of medical data, performing a data processing process on each of the plurality of medical data, and arranging and integrating each of the processed data to create a plurality of risk determination information; and a judgment unit comprising a storage unit and a prediction unit, wherein the plurality of risk determination information is stored in the storage unit, and the prediction unit performs estimation to generate a risk evaluation information according to the plurality of risk determination information.

Regarding the system of predicting risks with biomedical data, the plurality of data comprises a personal profile data, a personal test data, a personal examination data, a diagnosis data or a combination thereof.

Regarding the system of predicting risks with biomedical data, the data processing unit comprises: a data quality unit for performing an equalization judgment on each of the plurality of medical data to generate an equalization judgment information; an information expansion unit for performing data expansion according to the equalization judgment information to generate an expansion information; a blank data-entering unit for performing a data-entering rule judgment according to the expansion information to generate a data-entering information; and an information selecting unit for performing arrangement and integration according to the data-entering information to create a plurality of risk determination information.

Regarding the system of predicting risks with biomedical data, the equalization judgment involves performing a feature engineering judgment on each of the plurality of medical data to generate a feature numerical value row, cutting the feature numerical value row into a plurality of data subsets according to a feature information, calculating the plurality of data subsets to generate a feature value, and testing the feature value against a threshold value to generate the equalization judgment information.

Regarding the system of predicting risks with biomedical data, the feature engineering comprises a numerical data standardization, a wording encoding, a category encoding, a deep learning or a combination thereof.

Regarding the system of predicting risks with biomedical data, the data-entering rule judgment comprises a first rule judgment dedicated to tested expansion information and a second rule judgment dedicated to untested expansion information.

Regarding the system of predicting risks with biomedical data, the first rule judgment involves entering an interpolated value if a null value lies between two tests and entering an extrapolated value if the null value precedes or follows a test, when the expansion information is marked to indicate that it has ever undergone the test.

Regarding the system of predicting risks with biomedical data, the second rule judgment involves entering a numerical value of a related data subset when the expansion information is marked to indicate that it has never undergone the test.

To achieve at least the above objective, the present disclosure provides a method of predicting risks with biomedical data, comprising the steps of: receiving a plurality of data and then arranging and integrating each of the plurality of data to create a plurality of medical data, by the data collecting unit; receiving the plurality of medical data by the data processing unit, performing the data test on each of the plurality of medical data by the data processing unit, and performing an equalization judgment on each of the plurality of medical data by a data quality unit of the data processing unit to generate an equalization judgment information; performing data expansion according to the equalization judgment information by an information expansion unit of the data processing unit to generate an expansion information; performing a data-entering rule judgment according to the expansion information by a blank data-entering unit of the data processing unit to generate a data-entering information; performing arrangement and integration according to the data-entering information by an information selecting unit of the data processing unit to create a plurality of risk determination information; and performing estimation according to the plurality of risk determination information by the prediction unit to generate a risk evaluation information.

To achieve at least the above objective, the present disclosure provides a non-transient state computer-readable storage medium, for storing a plurality of codes, wherein, to execute the method of predicting risks with biomedical data, a processor executes, after the codes have been loaded to the processor, the codes to perform the steps of: receiving a plurality of data and then arranging and integrating each of the plurality of data to create a plurality of medical data, by the data collecting unit; receiving the plurality of medical data by the data processing unit, performing the data test on each of the plurality of medical data by the data processing unit, and performing a equalization judgment on each of the plurality of medical data by a data quality unit of the data processing unit to generate an equalization judgment information; performing data expansion on the equalization judgment information by an information expansion unit of the data processing unit to generate an expansion information; performing a data-entering rule judgment on the expansion information by a blank data-entering unit of the data processing unit to generate a data-entering information; performing arrangement and integration according to the data-entering information by an information selecting unit of the data processing unit to create a plurality of risk determination information; and performing estimation according to the plurality of risk determination information by the prediction unit to generate a risk evaluation information.

### BRIEF DESCRIPTION OF THE DRAWINGS

FIG. 1 is a schematic view of a system of predicting risks with biomedical data according to an embodiment of the present disclosure.

FIG. 2 is a schematic view of KS-test.

FIG. 3 is a schematic view of a VAE deep learning model.

### DETAILED DESCRIPTION OF THE INVENTION

To facilitate understanding of the object, characteristics and effects of this present disclosure, embodiments together with the attached drawings for the detailed description of the present disclosure are provided.

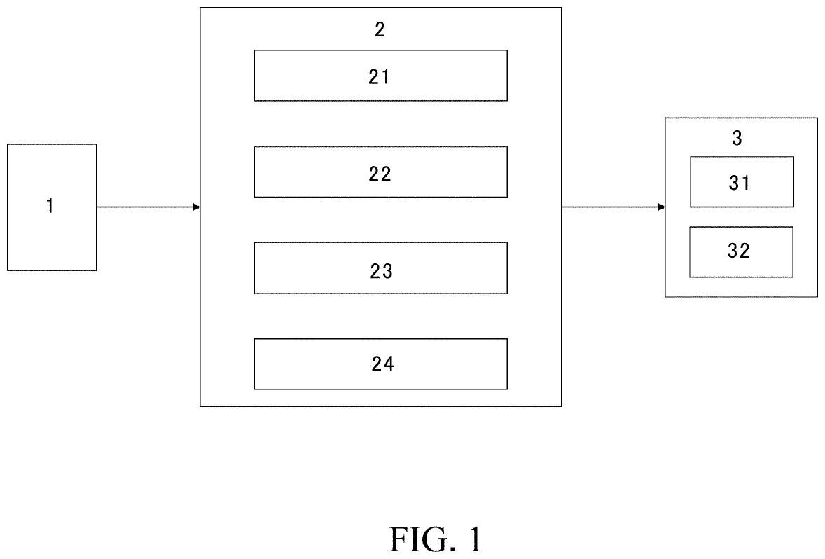

Referring to FIG. 1, there is shown a schematic view of a system of predicting risks with biomedical data according to an embodiment of the present disclosure. The system of predicting risks with biomedical data comprises a data collecting unit 1, data processing unit 2 and judgment unit 3. The data collecting unit 1 receives a plurality of data and then arranges and integrates each of the plurality of data to create a plurality of medical data. The plurality of data originates from medical record data kept by medical institutions. In this embodiment, the plurality of data comprises a personal profile data, a personal test data, a personal examination data, a diagnosis data or a combination thereof. In this embodiment, the personal profile data includes patients' gender, age, and registration date, as disclosed by a registration information system. The personal test data is obtained by analyzing specimens provided by patients, for example, blood sugar and blood lipids. The personal examination data is obtained by directly measuring the human body, for example, body height and body weight. The diagnosis data is derived from medical orders given by physicians. The personal profile data, personal test data, personal examination data and diagnosis data undergo arrangement and integration, including detection and correction of format mistakes, format conversion, data segmentation, and data recombination, to create medical data. The present disclosure is not restrictive of the aforesaid data arrangement and integration but refrains from losing important content information. The aforesaid data arrangement and integration is applicable to all the steps described hereunder. The types of the aforesaid personal data and the ways of creating medical data from the personal data constitute some aspects of this embodiment but place no limitations on any other embodiment.

In this embodiment, the data processing unit 2 receives the plurality of medical data and performs a data processing process on each of the plurality of medical data. The data processing process is described in detail later. The data processing unit 2 comprises a data quality unit 21, information expansion unit 22, blank data-entering unit 23 and information selecting unit 24. After undergoing a data processing process, each data undergoes arrangement and integration to create a plurality of risk determination information. The judgment unit 3 comprises a storage unit 31 and a prediction unit 32. The plurality of risk determination information is stored in the storage unit 31. The prediction unit 32 performs estimation according to the plurality of risk determination information to generate a risk evaluation information.

In this embodiment, the data collecting unit, data processing unit and prediction unit are each a computation device which executes a program, and the storage unit is a non-transient state storage medium. However, in a variant embodiment, the data collecting unit, data processing unit and prediction unit each comprise a combination of a processor and codes executable by the processor.

In this embodiment, the risk determination information is medical record data processed and sent from the data processing unit 2. In this embodiment, the risk evaluation information is an output value of the prediction unit 32 and is Softmax activation function of deep learning output layer.

The specific operation process flow of the data processing unit 2 and judgment unit 3 is generally described below.

The data processing process is carried out with the data quality unit 21, information expansion unit 22, blank data-entering unit 23 and information selecting unit 24 and involves: performing an equalization judgment on each of the plurality of medical data by the data quality unit 21 to generate an equalization judgment information and performing an equalization process on data to be equalized according to the equalization judgment information so as for different data to have a comparison standard in common; performing data expansion according to the equalization judgment information by the information expansion unit 22 to generate an expansion information; performing a data-entering rule judgment according to the expansion information by the blank data-entering unit 23 to generate a data-entering information; and performing arrangement and integration according to the data-entering information by the information selecting unit 24 to create a plurality of risk determination information. The equalization judgment, data expansion, data-entering rule judgment and risk determination information creation are described in detail below.

In this embodiment, the equalization judgment involves performing a feature engineering judgment on each of the plurality of medical data to generate a feature numerical value row, wherein the feature engineering comprises a numerical data standardization, a wording encoding, a category encoding, a deep learning or a combination thereof. The feature numerical value row is cut into a plurality of data subsets according to one or more feature information (for example, data source, examination year, patient gender, signs and symptoms, but the present disclosure is not limited thereto). Then, the plurality of data subsets are computed to generate a feature value. After that, the feature value is tested against a threshold value. Finally, the equalization judgment information is generated. The equalization judgment information is for use in comparing statistical distribution of the feature value in k data subsets and statistical distribution of the feature value in k0 data subsets (The comparison method for statistical distribution is described later.) If the statistical difference is greater than the threshold value, an equalization process will be required, otherwise the initial value will be retained. The equalization judgment information is representative median (M) and divergence (S) of feature numerical values within each subset. The equalization process corrects the median (M) and divergence (S) of specific feature values with the equation below,

$${x}_{new} = \frac{{x}_{old} - {M}_{x , k}}{{S}_{x , k}} \cdot {S}_{x , {k}_{0}} + {M}_{x , {k}_{0}}$$

where x_{old} denotes feature value before correction, x_{new}, denotes feature value after correction, k denotes x_{old} serial number of related data subset, k₀ denotes serial number of specific data subset. The median (M) is the average or median of a data subset. The divergence (S) is the standard deviation or divergence of a data subset. k₀ denotes data subsets in a specific year.

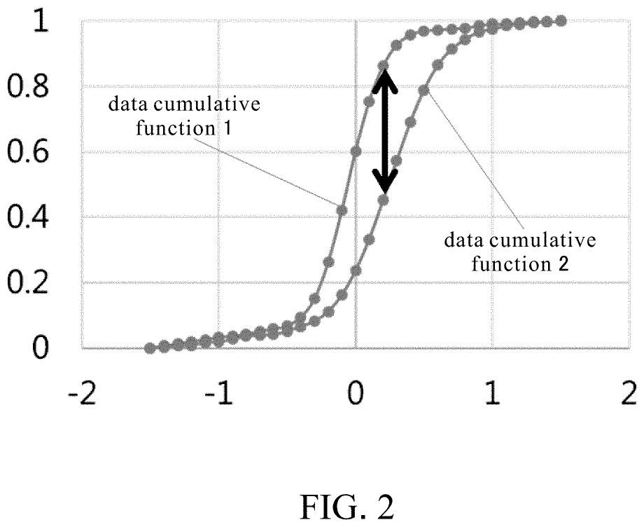

Regarding the equalization judgment, FIG. 2 is a schematic view of KS-test based on FIG. 1. When each of the plurality of medical data undergoes equalization judgment, not all the feature values have to be equalized, and its evaluation method involves comparing statistical distribution of a feature value within k data subsets and statistical distribution of the feature value within k0 data subsets. If the statistical difference is greater than a threshold value, an equalization process will be required, otherwise the initial value will be retained. The present disclosure involves performing quantitative analysis of data quality with Kolmogorov-Smirnov test (KS-test) by dividing feature values into an experiment group and a control group and then displaying the overlapped feature values on the coordinates after data cumulative function conversion. If the two groups are similar in data cumulative function quality (data cumulative function 1, data cumulative function 2), the largest difference value (also known as KS-test value) in data cumulative function between the two groups approximates to 0. Conversely, the greater the difference in data cumulative function quality between the two groups is, the closer is the largest difference value in data cumulative function between the two groups to 1. The method is advantageous in that it dispenses with the need to assume that the distribution function of data population is better than normal distribution of T-test, and thus the method applies to most medical numerical information. According to the present disclosure, KS-test value must be less than 0.1, otherwise the numerical value must be corrected with an equation.

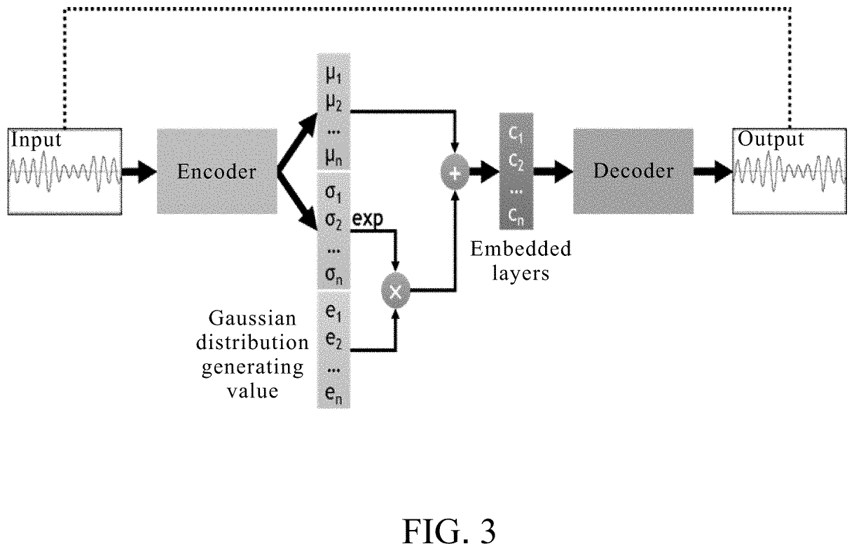

The plurality of medical data comprises quantitative medical data like systolic and diastolic blood pressure levels and blood sugar level and non-quantitative medical data like x-ray images and electrocardiogram signals. To enable the data quality unit 21 to perform data equalization on non-quantitative medical data, this embodiment provides a method of converting non-quantitative medical data into feature series for use by the data quality unit 21. Referring to FIG. 3, there is shown a schematic view of a VAE deep learning model. For example, non-quantitative signal data like x-ray images and electrocardiogram signals are converted into feature series with a variational autoencoder (VAE) in a deep learning model. In the course of data encoding, embedded layer series [c1, c2, cn] necessarily follows Gaussian distribution, and the series are decoded and restored to become input data. Since the embedded layers in VAE include feature series capable of restoring data, initial signal data can be replaced with feature series capable of restoring data, so as to correct the feature series which originates from the signal data and attain its equalization. In yet another preferred embodiment, correctable non-quantitative medical data further includes word embedding vectors. The word embedding vectors project a word or text to a word vector space with word frequency statistical methods, such as a neural network, to form a numerical vector v^{→}=[s_1,s_2, . . . ,s_n]. When a value s attains the aforesaid numerical correction standard, the value s (the value s is the series of one of the embedded layers in the neural network) must be corrected with an equation for use in correction of a specific feature value in the equalization process. The aforesaid technique can be used to convert verbal data, such as medical orders and nursing records, into the feature series for use by the data quality unit 21.

In this embodiment, the information expansion unit 22 performs data expansion according to the equalization judgment information to generate an expansion information. In particular, the data quality unit 21 equalizes a value or chooses to retain an initial value, so as to generate two types of judgment information: the equalized value and the initial value. The data quality unit 21 enters the equalized value or initial value into the information expansion unit 22 for data expansion.

The information expansion unit 22 expands information with one or more computation processes to generate new information. The computation processes involve applying clinical rules or mathematical equations, for example, body mass index, metabolism syndrome risks, and ten-year cardiovascular disease risks.

In this embodiment, the blank data-entering unit 23 performs a data-entering rule judgment according to the expansion information to generate a data-entering information. The data-entering rule judgment comprises a first rule judgment and a second rule judgment. The first rule judgment involves entering an interpolated value (for example, linear interpolated value, the nearest value) if a null value lies between two tests and entering an extrapolated value (for example, linear extrapolated value, the nearest value) if the null value precedes or follows a test, when the expansion information is marked to indicate that it has ever undergone the test. The second rule judgment involves entering a numerical value of a related data subset when the expansion information is marked to indicate that it has never undergone the test.

In this embodiment, the information selecting unit 24 performs arrangement and integration according to the data-entering information with statistical indexes and machine learning (i.e., according to an information selecting rule whereby a computer selects information automatically) to create a plurality of risk determination information. However, the data-entering information include quantitative information and non-quantitative information. The quantitative information include quantitative physiological numerical values (for example, body height, body weight and blood pressure), and its importance is subjectively determined by clinical experts or objectively sorted according to statistical indexes and machine learning. The non-quantitative information include non-quantitative physiological information (for example, electrocardiogram and electroencephalography), which clinical experts can only qualitatively determine; thus, non-quantitative information is converted into the feature series with deep learning, and then the feature series which originates from quantitative information and non-quantitative information is arranged and integrated according to the importance of feature numerical values with statistical indexes and machine learning to create a plurality of risk determination information. In this embodiment, the arrangement and integration is performed according to statistical indexes, machine learning, Pearson's correlation coefficient or decision tree analysis. In a variant embodiment, a plurality of risk determination information is created according to the importance of feature numerical values with any other tools, for example, support vector machine. The Pearson's correlation coefficient equation:

$$r = \frac{{\sum }_{i} ( {x}_{i} - \overline{x} ) \cdot ( {y}_{i} - \overline{y} )}{\sqrt{{\sum }_{i} {( {x}_{i} - \overline{x} )}^{2} \cdot {\sum }_{i} {( {y}_{i} - \overline{y} )}^{2} .}}$$

where xᵢ denotes the i^{th} feature numerical value, x̅ denotes the numerical average value of the feature, yᵢ denotes the i^{th} prediction target value, and y̅ denotes the numerical average value of the prediction target. The Pearson's correlation coefficient ranges from −1 to 1, wherein a positive value shows that x, y data distribution is positive correlated, and a negative value indicates negative correlation. The greater the absolute value is, the stronger the correlation is. The feature data is selected according to the resultant numerical value. The decision tree analysis evaluates data by decision tree theories and programs, such as random forest, LightGBM, and XGBoost. The decision tree analysis comprises judgment nodes. The nodes each have a feature, a judgment logic and a post-judgment resolution. The decision tree analysis requires plenty data and repeated computation to optimize configuration of the nodes with a view to maximizing overall accuracy. The node optimization process tends to use feature data with high resolution; thus, given appropriate decision tree training, a feature's importance is evaluated in the light of the quantity of the nodes of the feature. Therefore, a feature series which originates from quantitative information and non-quantitative information undergoes arrangement and integration with Pearson's correlation coefficient or decision tree analysis to create a plurality of risk determination information.

In this embodiment, the plurality of risk determination information undergoes estimation with the prediction unit 32 to generate a risk evaluation information. The estimation is carried out with deep neural networks (DNN), gated recurrent units (GRUs), and neural networks (NN) inside the time distributed wrapper. The plurality of risk determination information enters the deep neural networks. Then, early data has top priority to be processed, and 16 embedded layer numerical values are output. After the gated recurrent unit networks have received the data, the deep neural networks process the next time data. After that, the gated recurrent unit networks sequentially receive the embedded layer data. Previous neuron memory serves as a reference for each instance of receiving data and computation, and memory is transmitted to the next instance of computation. Upon computation of a plurality of time points, the final gated recurrent unit network outputs 64 numerical values to two neurons of a monolayer neural network. Softmax activation function outputs a risk evaluation information (diseases' negative rate and positive rate prediction values).

Yet another preferred embodiment provides a method of predicting risks with biomedical data. In this embodiment, the method of predicting risks with biomedical data is executed with the system of predicting risks with biomedical data. The method of predicting risks with biomedical data comprises the steps of: receiving a plurality of data by the data collecting unit 1; arranging and integrating each of the plurality of data by the data collecting unit 1 to create a plurality of medical data; receiving the plurality of medical data by the data processing unit 2; and performing a data processing process on each of the plurality of medical data by the data processing unit 2. The data processing process involves performing an equalization judgment on each of the plurality of medical data by a data quality unit 21 of the data processing unit 2 to generate an equalization judgment information, performing data expansion according to the equalization judgment information by an information expansion unit 22 of the data processing unit 2 to generate an expansion information, performing a data-entering rule judgment according to the expansion information by a blank data-entering unit 23 of the data processing unit 2 to generate a data-entering information, performing arrangement and integration according to the data-entering information by an information selecting unit 24 of the data processing unit 2 to create a plurality of risk determination information, and performing estimation according to the plurality of risk determination information by the prediction unit 32 to generate a risk evaluation information.

A further preferred embodiment provides a non-transient state computer-readable storage medium, for storing a plurality of codes, wherein, to execute the method of predicting risks with biomedical data, a processor executes, after the codes have been loaded to the processor, the codes to perform the steps of: receiving a plurality of data and then arranging and integrating each of the plurality of data to create a plurality of medical data, by the data collecting unit; receiving the plurality of medical data by the data processing unit, performing the data test on each of the plurality of medical data by the data processing unit, and performing a equalization judgment on each of the plurality of medical data by a data quality unit of the data processing unit to generate an equalization judgment information; performing data expansion according to the equalization judgment information by an information expansion unit of the data processing unit to generate an expansion information; performing a data-entering rule judgment according to the expansion information by a blank data-entering unit of the data processing unit to generate a data-entering information; performing arrangement and integration according to the data-entering information by an information selecting unit of the data processing unit to create a plurality of risk determination information; and performing estimation according to the plurality of risk determination information by the prediction unit to generate a risk evaluation information.

The system of predicting risks with biomedical data corrects data and enters a missing value with an algorithm to render the data consistent and enhance prediction accuracy. Therefore, the system of predicting risks with biomedical data eliminates the effect of bad data on prediction accuracy of artificial intelligence models and thereby enhances the accuracy in predicting disease risks from personal health records.

While the present disclosure has been described by means of specific embodiments, numerous modifications and variations could be made thereto by those skilled in the art without departing from the scope and spirit of the present disclosure set forth in the claims.

What is claimed is:

1. A system of predicting risks with biomedical data, comprising:
a data collecting unit for receiving a plurality of data and then arranging and integrating each of the plurality of data to create a plurality of medical data;
a data processing unit for receiving the plurality of medical data, performing a data processing process on each of the plurality of medical data, and arranging and integrating each of the processed data to create a plurality of risk determination information; and
a judgment unit comprising a storage unit and a prediction unit, wherein the plurality of risk determination information is stored in the storage unit, and the prediction unit performs estimation to generate a risk evaluation information according to the plurality of risk determination information.

2. The system of predicting risks with biomedical data according to claim 1, wherein the plurality of data comprises a personal profile data, a personal test data, a personal examination data, a diagnosis data or a combination thereof.

3. The system of predicting risks with biomedical data according to claim 1, wherein the data processing unit comprises:
a data quality unit for performing an equalization judgment on each of the plurality of medical data to generate an equalization judgment information;
an information expansion unit for performing data expansion according to the equalization judgment information to generate an expansion information;
a blank data-entering unit for performing a data-entering rule judgment according to the expansion information to generate a data-entering information; and
an information selecting unit for performing arrangement and integration according to the data-entering information to create a plurality of risk determination information.

4. The system of predicting risks with biomedical data according to claim 3, wherein the equalization judgment involves performing a feature engineering judgment on each of the plurality of medical data to generate a feature numerical value row, cutting the feature numerical value row into a plurality of data subsets according to a feature information, calculating the plurality of data subsets to generate a feature value, and testing the feature value against a threshold value to generate the equalization judgment information

5. The system of predicting risks with biomedical data according to claim 4, wherein the feature engineering comprises a numerical data standardization, a wording encoding, a category encoding, a deep learning or a combination thereof.

6. The system of predicting risks with biomedical data according to claim 3, wherein the data-entering rule judgment comprises a first rule judgment dedicated to tested expansion information and a second rule judgment dedicated to untested expansion information.

7. The system of predicting risks with biomedical data according to claim 6, wherein the first rule judgment involves entering an interpolated value if a null value lies between two tests and entering an extrapolated value if the null value precedes or follows a test, when the expansion information is marked to indicate that it has ever undergone the test.

8. The system of predicting risks with biomedical data according to claim 6, wherein the second rule judgment involves entering a numerical value of a related data subset when the expansion information is marked to indicate that it has never undergone the test.

9. A method of predicting risks with biomedical data, using the system of claim 1, the method comprising the steps of:
receiving a plurality of data and then arranging and integrating each of the plurality of data to create a plurality of medical data, by the data collecting unit;
receiving the plurality of medical data by the data processing unit, performing the data test on each of the plurality of medical data by the data processing unit, and performing an equalization judgment on each of the plurality of medical data by a data quality unit of the data processing unit to generate an equalization judgment information;
performing data expansion according to the equalization judgment information by an information expansion unit of the data processing unit to generate an expansion information;
performing a data-entering rule judgment according to the expansion information by a blank data-entering unit of the data processing unit to generate a data-entering information;
performing arrangement and integration according to the data-entering information by an information selecting unit of the data processing unit to create a plurality of risk determination information; and
performing estimation according to the plurality of risk determination information by the prediction unit to generate a risk evaluation information.

10. A non-transient state computer-readable storage medium, for storing a plurality of codes, wherein, after the codes have been loaded to a processor, the processor executes the codes to perform the steps of:
receiving a plurality of data and then arranging and integrating each of the plurality of data to create a plurality of medical data, by the data collecting unit;
receiving the plurality of medical data by the data processing unit, performing the data test on each of the plurality of medical data by the data processing unit, and performing a equalization judgment on each of the plurality of medical data by a data quality unit of the data processing unit to generate an equalization judgment information;
performing data expansion according to the equalization judgment information by an information expansion unit of the data processing unit to generate an expansion information;
performing a data-entering rule judgment according to the expansion information by a blank data-entering unit of the data processing unit to generate a data-entering information;
performing arrangement and integration according to the data-entering information by an information selecting unit of the data processing unit to create a plurality of risk determination information; and
performing estimation according to the plurality of risk determination information by the prediction unit to generate a risk evaluation information.
